(19) United States
(12) Patent Application Publication
(10) Pub. No.: US 2023/0236183 A1
(43) Pub. Date: Jul. 27, 2023

(54) NANO-POROUS CERAMIC FILMS FOR HIGH DENSITY BIOASSAY MULTIPLEXED ARRAYS

(71) Applicant: NANOPEC, INC., TUCSON, AZ (US)

(72) Inventor: Mario Blanco, Tucson, AZ (US)

(73) Assignee: NANOPEC, INC., TUCSON, AZ (US)

(21) Appl. No.: 18/011,312

(22) PCT Filed: Jun. 16, 2021
(86) PCT No.: PCT/US2021/037613

§ 371 Date: Dec. 19, 2022

Related U.S. Application Data
(60) Provisional application No. 63/040,245, filed on Jun. 17, 2020.

(51) Int. Cl. G01N 33/543; G01N 33/542

(52) U.S. Cl. CPC G01N 33/54373; G01N 33/542

(57) ABSTRACT

A nano-porous structure substrate forming assays occupying no more than one square micron. The assays are comprised of bundled cylindrical nano-pores that act as vessels that can house reagents for a single specific bioassay. A substrate of only a few square centimeters can accommodate 100,000 to 1,000,000 individual bioassays. The substrate may be doped with fluorescent enhancement centers to increase the signal to noise ratios or be surface modified with grafting compounds such as universal linkers, silane coupling agents, antigens and antibodies, or gene sequences.

### BACKGROUND OF THE INVENTION

### 1. Field of the Invention

The present invention relates to ceramic films and, more specifically, to a nano-porous structure substrate for performing a multitude of individual assays.

### 2. Description of the Related Art

The field of medicine continues to evolve at a high speed. Even medical experts are not able to keep up with their individual fields with the amount of new scientific findings in the causes, evolution and treatment of existing and new health conditions. This is true for inherited (genetic), infectious diseases and cancer. Personalized medicine has become increasingly important in effective patient care. Big data analysis and artificial intelligence combined with genomics, epigenetics and proteomics are revolutionizing the field of medicine. From diagnostics to treatment these tools will make early disease detection, prevention and treatment more effective.

Massive amounts of data on a single individual are now available or in the development pipeline. Perhaps a single blood test might be sufficient to obtain all the necessary information (liquid biopsies) to diagnose and treat a patient in the near future. Artificial intelligence will link big data to diagnosis and treatment with or without the need for training sets and the practice of medicine will change forever. Genomics has made tremendous progress in the last decade, reaching now the stage where full gene sequencing of an individual patient is possible at a relatively affordable cost. As a result, there is a need for more effective means to gather such data.

Focused, flexible multiplexing of one to 500 analytes meets the needs of a wide variety of applications - protein expression profiling, focused gene expression profiling, autoimmune disease, genetic disease, molecular infectious disease, and human leucocyte testing (HLA). Currently available, these analyte bioassays do not provide capabilities that are anything close to what might be needed for the characterization of the 25,000 genes a human genome contains, so there is need for improvement in technology to bring analyte bioassays to the same level as genomics where large, high quality data sets numbering in the tens of thousands, rather than just the hundreds, are possible.

### BRIEF SUMMARY OF THE INVENTION

The present invention offers a nano-porous structure substrate where individual assays occupying no more than one square micron can be accomplished, thereby allowing for tens of thousands of assays to be performed at one time. Due to the nature of the pores, i.e., straight, not cross over, a single square micron contains approximately 100 bundled nano-vessels where reagents can be housed and isolated for a single specific bioassay. Typical dimensions of these substrates are a few square centimeters, which will still provide a sufficient surface area to accommodate 100,000 to 1,000,000 different bioassays due to the nature of the pores. The substrate may or may not be doped with fluorescent enhancement FRET centers to increase the signal to noise ratios. Reading by opto-electronic means is available with off the shelf charged-coupled device (CCD) cameras and optical fibers. The nano-porous structure substrate for such an assay is the nano-structured ceramic film of the present invention.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1A is a schematic of a nano-structured ceramic film according to the present invention;

FIG. 1B is a schematic of pore size distribution of a nano-structured ceramic film according to the present invention;

FIG. 1C is a graph of pore size distribution of a nano-structured ceramic film according to the present invention

FIG. 2 is a schematic of an exemplary circular piece of ceramic (lentil) containing a plurality of individual assays on a nano-structured ceramic film according to the present invention;

FIG. 3 is a flow diagram illustrates how the information on a single lentil containing 10 x 10 bioassay array may be processed according to the present invention; and

FIG. 4 is a schematic of optoelectronic equipment assembly for reading an array of assays formed by a nano-structured ceramic film according to the present invention.

### DETAILED DESCRIPTION OF THE INVENTION

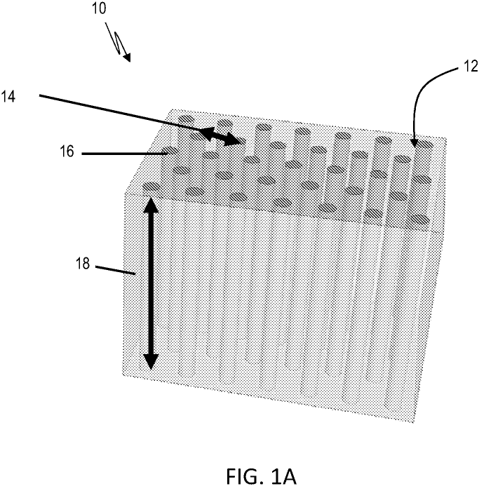

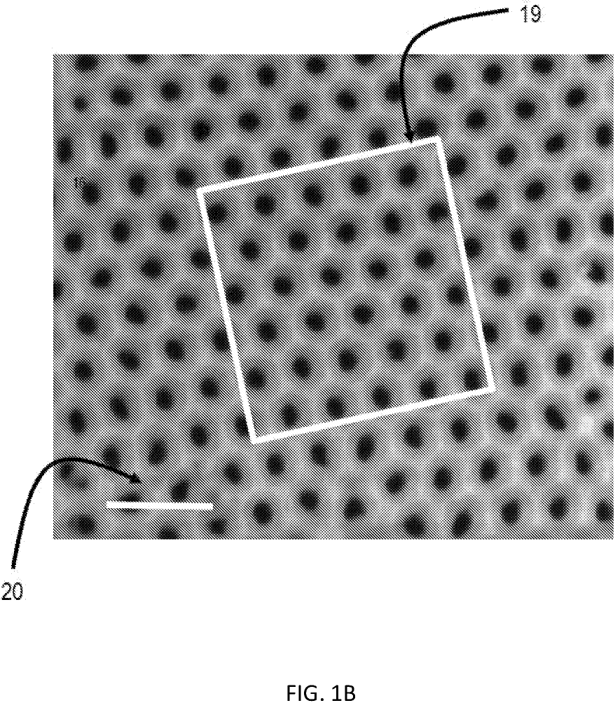

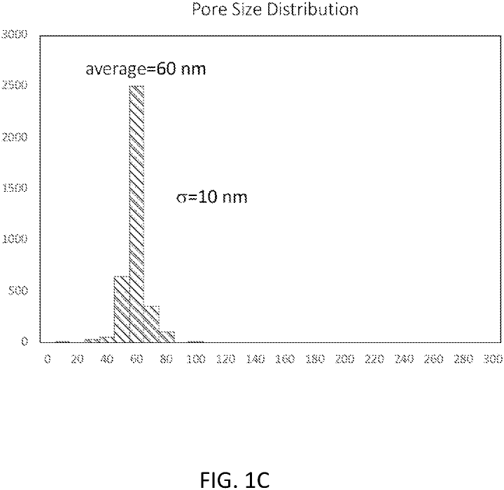

Referring to the figures, wherein like numeral refer to like parts throughout, there is seen in FIGS. 1 a nano-structured ceramic film 10 having a plurality of pores 12 following typical pore size distribution. More specifically, plurality of pores 12 are arranged in a lattice having a lattice constant 14 of up to 500 nanometers, wherein each of the pores has a diameter 16 of up to 400 nanometers and a length of up to 100,000 nanometers and can serve as an individual bioassay or bioassay location. As seen in FIG. 1B, a scanning electron micrograph of the invention shows a typical section 19 containing 30 pores in .225 micron². The white bar 20 is 200 nanometers long. Up to 133 pores per square micron can be located with an interpore distance of 82 nanometers, which can be controlled through synthesis conditions for the nano-porous ceramic substrate used in the present invention. These include the pore diameter, pore ordered distance, pore depth. Pores can be through pores or closed pores with an aluminum backing as shown. Typical pore size distribution obtained from scanning electron microscopy for the ceramic disc invention is shown in FIG. 1C. The standard deviation of the pore distribution is approximately σ = 10 nm.

Nano-structured ceramic films according to the present invention may be manufactured by degreasing an aluminum plate using a degreasing solution, electropolishing the aluminum plate after degreasing with an electropolishing solution that is free of perchloric acid and chromic acid, pre-anodizing the aluminum plate after electropolishing with an anodization acid solution for a first predetermined time period, anodizing the aluminum plate after electropolishing with the anodization acid solution for a second predetermined time period to form an anodized membrane on the aluminum plate, separating the anodized membrane from the aluminum plate, and cleaning the anodized membrane. The step of degreasing the aluminum plate may comprise immersing the aluminum plate in acetone and ethanol. The step of electropolishing the aluminum plate may comprise bathing the aluminum plate in a bath of phosphoric acid. The bath of phosphoric acid may comprise from about 30 percent to about 95 percent of phosphoric acid and from about 5 percent to about 70 percent of polyethylene glycol. The step of bathing the aluminum plate in a bath of phosphoric acid is performed at a voltage of from about 15 to 100 volts, at a temperature from about 35° C. to about 65° C., and at a current density of from about 3 mA/cm² to about 160 mA/cm². The step of pre-anodizing the aluminum plate may comprise immersing the aluminum plate in an anodizing acid for between five and twenty minutes. The step of pre-anodizing the aluminum plate may comprise immersing the aluminum plate in an anodizing acid for up to twenty four hours. The step of separating the anodized membrane from the aluminum plate may comprise the step of performing soluble membrane separation. The step of performing soluble membrane separation may comprise immersing the aluminum plate in sulfuric acid. The step of cleaning the anodized membrane may comprise submerging the anodized membrane in a low concentration phosphoric acid solution (2-6%) for five to fifteen minutes followed by sonicating in ultra-pure water for up to 15 minutes.

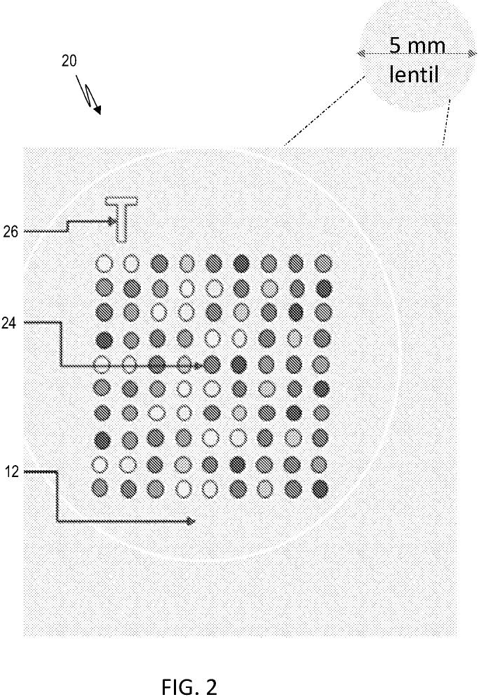

Referring to FIG. 2, the nano-porous ceramic film 10 can be laser cut defect free (no microcracks) in different sizes and shapes, such as being configured into a lentil 22 having a plurality of discrete bioassay regions 24, each of which can include multiple individual assays in combination, for use in a multiplexed bioassay 20. A lentil 12 that is 5 mm in diameter is illustrated for example. An alignment mark 26 may be included on lentil 12 for automated processing and can help re-orient the information for automated optical reading. Any other alignment symbol including a QR code can be employed for this purpose. Nano-structured ceramic film 10 can be doped with FRET centers. Pore walls composition is anodized aluminum oxide (Al₂O₃) ceramic. Fluorescence resonance energy transfer (FRET) centers include chelated metal ions. Each lentil may contain a positional array (Xn, Yn) with individual reagents to conduct a single bioassay.

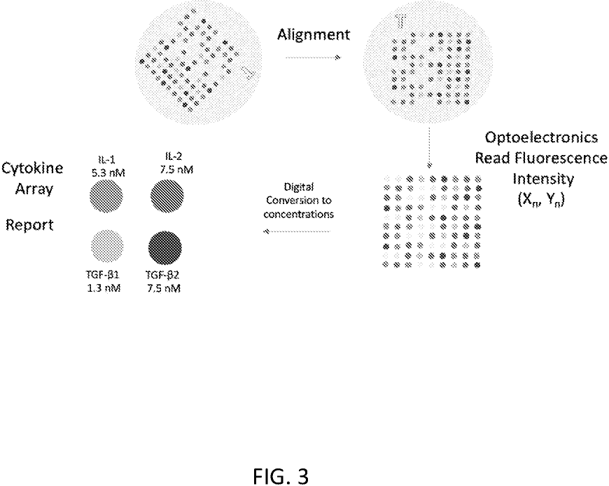

Referring to FIG. 3, lentil 12 may be processed with software to extract the bioassay information, after reagents have been placed and incubated with a patient sample. For example, as seen in FIG. 3, a simple (10x10) bioassay array is used to illustrate the information on a single lentil may be processed by the software. After a picture of the emitted light is taken, with a high-resolution CCD camera, the digitized image is rotated by the analysis software and re-oriented. The positions of each of the individual bioassays are then used to identify the biologic (peptide, protein, DNA or RNA oligonucleotide, hormone, etc) being identified and measure at each of the (Xn, Yn) positions. The intensity of the color or fluorescence signal is then used to convert it to a concentration and a report of the full array is produced as a bioassay report and made available for human or digital (AI) processing.

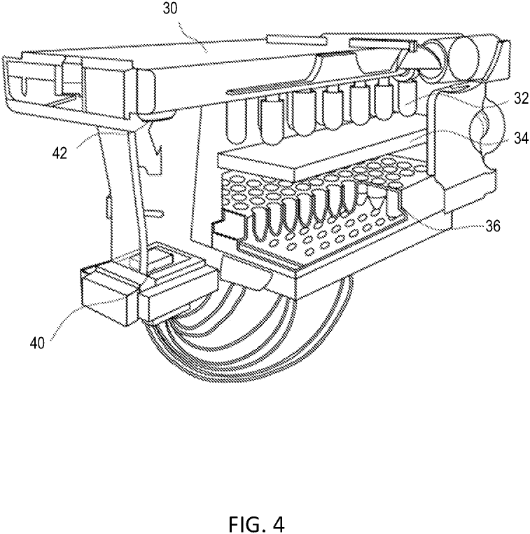

Referring to FIG. 4, an illustrative example of the optoelectronic equipment necessary to read and process the information on lentil 12 when used as a bioassay substrate is shown. The basic equipment comprises a high-resolution charged coupled device (CCD) camera 30 suspended by a mount 42 positioned over a microplate 36 to capture a digital image of a large number of assays for further software processing. Lenses 32 and filters 34 may be positioned between camera 30 and microplate 36 to enhance the digital image capture, and an optical fiber bundle 40 may be used to capture images of individual wells of microplate 36 m which can contain, 96, 384, or even a higher number of wells (with a corresponding number of optical fibers to provide bottom reads). Filters 34 are optional and may be selected depending on the dyes used by the particular individual bioassay.

A photo-active enhanced fluorescence ceramic film according to the present invention can be used in all forms of multiplex bioassays. Detection by fluorescence or chromogenic means can be employed. Uses include clinical in vitro diagnostics but also as a research tool for medical investigations such as genetic mutations or deletions, infectious diseases, vaccine development, and cancer diagnoses. The present invention provides for extremely high density of bioassays due to the nano-porous structure of the film as well the addition of linker molecules for the attachment of any biological compound of interest. Fast detection is also possible as the present invention only requires a limited number of preparatory steps (reagent reaction times) and the data acquisition rates are measured in microseconds rather than minutes in standard microfluidic bead flow multiplexing systems that may require microsecond residence times. Low detection volumes are also possible as enhancement occurs within the ceramic discs, which for a typical bundled pore dot (1 square micron) can be as little as 10 to 1,000 femtoliters, requiring low reagent use per bioassay. Signal to noise enhancement by photonics effects (pore geometry) and optionally by FRET fluorescence enhancement with doped species embedded in the ceramic film are also possible. The present invention further provides an easy means to keep track of individual bioassay by automated software localization means and permanent recording of results that are otherwise readily available data for human or digital (AI) analysis. Optionally, a second porous ceramic film loaded with capture reagents can be placed over the bioassay lentil to eliminate specific proteins such as, without limitation, human antibodies, which can be a main source of non-specific binding interference in other multiplexing bioassays. Due to the limits presented by sub-wavelength resolution, an individual bioassay should be extended beyond the longest possible visible or near IR optical detection wavelengths. In addition, due to current repeatability in deposition locations, individual biossays must be spaced out to avoid overlaps of reagent zones.

The present invention may include chelated metal ion FRET centers doped within the nano-structure ceramic film as the means for fluorescence enhancement. Typical fluorescence lifetimes in bioassay detection are measured in the range of 10 to 100 nanoseconds. Enabled by chelated metal ion FRET centers doped within the nano-structure ceramic film, the present invention can provide long lasting, several micro-seconds, fluorescence signals. According to the present invention, the chelated metal ion centers may include transition metals and lanthanides embedded in the ceramic film during fabrication at low concentration (doping). Due to the absence of quenching solvent water molecules, the FRET centers can remain in a long-lived triplet state (on the order of tens of microseconds) when excited by an external source. This in turn makes them a convenient energy reservoir - the FRET centers act as non-radiative (electronic) donors, amplifying the fluorescence signal strength by recharging the fluorescent probes over a period of time comparable to one thousand times their normal fluorescent lifetimes (tens of nanoseconds). This amplification effect is only effective on a nanometric-scale, which has been established to be around 10 nm. This is sufficiently large to accommodate all the reagents inside the interior pore surface of the nano-porous ceramic used as substrate for the multiplexing bioassay array. The substrate may also be surface modified with grafting compounds such as universal linkers, silane coupling agents, antigens and antibodies, or even gene sequences.

Preliminary results collected using RT-PCR DNA probes enhanced by FRET electro-optical means have shown fluorescence multiplication values that are improved by greater than 1,000 percent as compared to standard (glass, plastic) substrates, with only a minor (< 5%) increase in background noise. Such fluorescence enhancement corresponds to improvements of signal-to-noise ratio (SNR) of greater than 10.

The present invention may further include the means to orient and identify individual bioassays through software processing. Reports may be generated for human or digital (AI) processing.

Considering a 750 nm wavelength read, the present invention can provide a resolution of 0.75 micrometers. This translates to an assay deposition repeatability of 1 to 5 micrometers with a spacing of 2 to 5 micrometers. As a result, 100,000 to 10,000,000 individual assays may be provided per square centimeter of nano-structured ceramic film according to the present invention.

What is claimed is:

1. A multiplexed bioassay system, comprising a lentil formed from a ceramic film of anodized aluminum oxide (Al₂O₃) and having a plurality of pores arranged in a lattice having a lattice constant of up to 500 nanometers, wherein each of the pores has a diameter of up to 400 nanometers and a length of up to 100,000 nanometers.

2. The multiplexed bioassay system of claim 1, wherein the plurality of pores have an average diameter of 60 nanometers.

3. The multiplexed bioassay system of claim 2, wherein the lentil has a diameter of five millimeters.

4. The multiplexed bioassay system of claim 3, wherein the ceramic film is doped with a FRET center.

5. The multiplexed bioassay system of claim 4, wherein the FRET center comprises a chelated metal ion.

6. The multiplexed bioassay system of claim 5, further comprising a microplate having a plurality of wells, wherein each of the plurality of wells include the lentil.

7. The multiplexed bioassay system of claim 5, wherein the lentil includes a surface treatment selected from the group consisting of silane coupling agents, linkers for peptide synthesis, linkers for nucleic acid oligonucleotides synthesis or grafting, linkers for peptides, linkers for proteins, linkers for antibodies, and linkers for genes.

8. The multiplexed bioassay system of claim 7, a charge-coupled device camera positioned to capture a digital image of the plurality of wells.

9. A method of providing a multiplexed bioassay system, comprising the steps of:
preparing a ceramic film from anodized aluminum oxide (Al₂O₃) to have a plurality of pores having a lattice constant of up to 500 nanometers, wherein each of the pores has a diameter of up to 400 nanometers and a length of up to 100,000 nanometers; and
laser cutting the ceramic film to form at least one lentil.

10. The method of claim 9, wherein the plurality of pores have an average diameter of 60 nanometers.

11. The method of claim 10, wherein the lentil has a diameter of five millimeters.

12. The method of claim 11, wherein the ceramic film is doped with a FRET center.

13. The method of claim 12, wherein the FRET center comprises a chelated metal ion.

14. The method of claim 13, further comprising the step of providing a surface treatment on the lentil, wherein the surface treatment is selected from the group consisting of silane coupling agents, linkers for peptide synthesis, linkers for nucleic acid oligonucleotides synthesis or grafting, linkers for peptides, linkers for proteins, linkers for antibodies, and linkers for genes.

15. The method of claim 14, further comprising the steps of:
providing a microplate having a plurality of wells; and
positioning each of the plurality of lentils in a corresponding one of the plurality of wells.

16. The method of claim 15, further comprising the step of positioning a CCD camera to capture a digital image of the plurality of wells having the plurality of lentils positioned therein.
